(19) United States
(12) Patent Application Publication
(10) Pub. No.: US 2005/0032892 A1
(43) Pub. Date: Feb. 10, 2005

(54) Compositions, kits, and methods for treating gastrointestinal conditions

(75) Inventors: Gary Robert Kelm, Cincinnati, OH (US); Jeffrey Warren Clymer, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/866,623

(22) Filed: Jun. 12, 2004

Related U.S. Application Data
(60) Provisional application No. 60/493,400, filed on Aug. 7, 2003.

(51) Int. Cl. A61K 31/22; A61K 31/20

(52) U.S. Cl. 514/546; 514/560

(57) ABSTRACT

Disclosed are compositions, kits, and methods which are useful for the treatment of gastrointestinal conditions in mammals. In one embodiment, the disclosure is directed to a method of treating a gastrointestinal condition in a mammal in need of such treatment, wherein the method comprises administering to the mammal a composition comprising a non-glyceryl ester of a long chain fatty acid. In another embodiment, the invention is directed to a method of treating a gastrointestinal condition in a mammal in need of such treatment, wherein the method comprises administering to the mammal a bismuth component and a long chain fatty acid component. In yet another embodiment, the disclosure is directed to a kit comprising a composition comprising a non-glyceryl ester of a long chain fatty acid; and information that the composition is useful for the treatment of a gastrointestinal condition in mammals. In still another embodiment, the invention is directed to a kit comprising a long chain fatty acid component, a bismuth component, and information that the composition is useful for the treatment of a gastrointestinal condition in mammals. In yet another embodiment, the disclosure is directed to a composition comprising a long chain fatty acid component selected from the group consisting of long chain fatty acids, non-glyceryl esters of long chain fatty acids, and mixtures thereof, and a bismuth component.

### CROSS REFERENCE TO RELATED APPLICATION

This application claims priority under Title 37, United States Code 119(e) from Provisional Application Ser. No. 60/493,400 filed Aug. 7, 2003.

### FIELD OF THE INVENTION

The present invention is directed to compositions, methods, and kits which are useful for the treatment of gastrointestinal conditions, for example, diarrhea.

### BACKGROUND OF THE INVENTION

Various gastrointestinal conditions, including diarrhea, can result from a variety of physiological disorders including bacterial or parasitic infections, disease or debilitation of organs such as liver, adrenal and others, and diet, as well as conditions such as irritable bowel syndrome or other like disorders. Diarrhea may also be symptom of a gastrointestinal condition and not itself a disorder. Chronic diarrhea is generally due to intestinal hypermotility and rapid transport. It may also be due to, or accompanied by, hypersecrection of acid gastric juices or decreased reabsorption and may, in some instances, be associated with emotional tension and psychological conflict or stress.

Antidiarrheal therapies are, of course, well-known in the medicinal arts and take various forms. In particular, there are a variety of products known which act systemically to provide antidiarrheal effects when administered in a manner which will enable the therapy to be take into the system at effective therapeutic levels. There is, however, a continuing need for additional effective antidiarrheal therapies.

One approach to antidiarrheal therapy has recently been proposed. This therapy, described in for example U.S. Pat. Nos. 5,977,175 and 6,558,708; U.S. Patent Application Publication Nos. US 2002/0094346 and US 2002/0039599; and WO 01/76631, suggests the use of “active lipids,” meaning those lipids which are digested or substantially digested molecules having a structure and function substantially similar to a hydrolyzed end-product of fat digestion. Examples of such active lipids are molecules such as glycerol and fatty acids.

A review of the literature suggests that the efficacy of this proposed therapy may be due to the putative ability of the active lipid to induce the release of pancreatic polypeptide YY (PYY) from the distal small intestine. PYY appears to be primarily responsible for the physiological responses that mitigate diarrhea, and its release is in turn dependent upon cholecystokinin (CKK) release from the proximal small intestine and/or direct contact of nutrients with the small intestine.

Unfortunately, the use of such active lipids in orally acceptable compositions may be impractical. Having extensive experience with fatty acid esters, the present inventors have found that these esters are much more aesthetically acceptable, particularly based on their organoleptic properties, relative to their corresponding fatty acids. However, in view of the literature, it may be counter-intuitive that such fatty acid esters would have similar or even improved antidiarrheal performance relative to these corresponding fatty acids, particularly since it has been suggested that the fatty acids have enhanced potency relative to triacylglycerides. Notwithstanding, it is the present inventors' surprising discovery that such fatty acid esters may be useful for the treatment of gastrointestinal conditions such as diarrhea, thereby providing a practical, consumer-acceptable route toward the effective treatment of these conditions.

It is with this background that the present invention is described with further detail, as follows:

### SUMMARY OF THE INVENTION

The present invention is directed to compositions, kits, and methods which are useful for the treatment of gastrointestinal conditions in mammals.

In one embodiment, the invention is directed to a method of treating a gastrointestinal condition in a mammal in need of such treatment, wherein the method comprises administering to the mammal a composition comprising a non-glyceryl ester of a long chain fatty acid. In another embodiment, the invention is directed to a method of treating a gastrointestinal condition in a mammal in need of such treatment, wherein the method comprises administering to the mammal a bismuth component and a long chain fatty acid component.

In yet another embodiment, the invention is directed to a kit comprising a composition comprising a non-glyceryl ester of a long chain fatty acid; and information that the composition is useful for the treatment of a gastrointestinal condition in mammals. In still another embodiment, the invention is directed to a kit comprising a long chain fatty acid component, a bismuth component, and information that the composition is useful for the treatment of a gastrointestinal condition in mammals.

In yet another embodiment, the invention is directed to a composition comprising a long chain fatty acid component selected from the group consisting of long chain fatty acids, non-glyceryl esters of long chain fatty acids, and mixtures thereof, and a bismuth component.

These and other embodiments are described in further detail herein.

### DETAILED DESCRIPTION OF THE INVENTION

Various documents including, for example, publications and patents, are recited throughout this disclosure. All such documents are hereby incorporated by reference.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments and/or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

### Definitions

As used herein, “companion animal” means a domestic animal. Preferably, “companion animal” means a domestic dog, cat, rabbit, ferret, horse, cow, or the like. Most preferably, “companion animal” means a domestic dog or cat.

As used herein, the term “safe and effective” means effective for the treatment of a gastrointestinal condition in a mammal (preferably a human or companion animal) without undue adverse side effects (such as toxicity, irritation, or allergic response), commensurate with a reasonable benefit/risk ratio when used in the manner of this invention.

### Compositions Employed in the Present Invention

The present invention is directed to compositions, kits, and methods that are suitable for treating a gastrointestinal condition in mammals. For example, kits and methods employed herein may utilize a composition comprising a non-glyceryl ester of a long chain fatty acid, which is a component further described herein. As another example, certain optional compositions employed herein may comprise a fatty acid component comprising a long chain fatty acid component selected from the group consisting of long chain fatty acids, non-glyceryl esters of long chain fatty acids, and mixtures thereof, as well as a bismuth component. Other components of various scope which may be used herein are described as follows:

The Long Chain Fatty Acid Component

The long chain fatty acid component (herein often referenced as “LCFA”) which is used in various embodiments herein is selected from the group consisting of long chain fatty acids, long chain fatty acid salts, non-glyceryl esters of long chain fatty acids, and mixtures thereof. As used herein, the LCFA contains a fatty acid chain, or wherein the fatty acid material is a fatty acid ester, contains a fatty acid chain and an ester chain. Thus, wherein the LCFA is a fatty acid, the material may be depicted as follows:
R—COOH
wherein “R” is the fatty acid chain which is a saturated or unsaturated chain having from about 10 to about 24 carbon atoms, and wherein “COOH” is a carboxylic acid moiety. More preferably, “R” is a saturated or unsaturated chain having from about 12 to about 24, most preferably from about 16 to about 18 carbon atoms. Also preferably, the fatty acid chain contains from 0 to about 3 double bonds. Most preferably, the fatty acid chain is unsaturated, in particular having one or two double bonds.

Wherein the fatty acid material is a long chain fatty acid salt, the fatty acid material is preferably acid-soluble, i.e., insoluble at a pH of greater than about 4 or soluble in 1N hydrochloric acid. This is particularly preferred for purposes of including the fatty acid material in a suspension or emulsion matrix. A preferred long chain fatty acid salt is a carnitine salt of a long chain fatty acid described herein.

Wherein the fatty acid material is a non-glyceryl ester of a fatty acid (sometimes referenced as a “non-glyceryl ester thereof”), the material may be depicted as follows:
R—COOR′

Wherein R is the fatty acid chain as defined above, and R′ is the ester chain, with the carboxylate moiety “COO” linking the two together. The ester chain is a straight or branched chain of carbon atoms that is hydrolyzable in the presence of mammalian digestive enzymes, preferably human digestive enzymes, and typically contains about 8 carbon atoms or less. The ester chain more preferably contains from 1 to about 5 carbon atoms and, again, may be a straight (for example, n-propyl) or branched (for example, iso-propyl) chain. Highly preferred ester chains include those that form methyl esters (i.e., R′ is —CH₃), ethyl esters, n-propyl esters, iso-propyl esters, n-butyl esters, iso-butyl esters, and mixtures thereof. Those ester chains that form ethyl esters are particularly preferred.

In a preferred embodiment of the present invention, the LCFA is selected from lauric acid, lauroleic acid, myristic acid, myristoleic acid, pentadecanoic acid, palmitic acid, palmitoleic acid, margaric acid, stearic acid, dihydroxystearic acid, oleic acid, ricinoleic acid, elaidic acid, linoleic acid, alpha-linolenic acid, dihomogamma-linolenic acid, eleostearic acid, licanic acid, arachidonic acid, arachidic acid, eicosenoic acid, eicosapentaenoic acid, behenic acid, erucic acid, docosahexaenoic acid, lignoceric acid, non-glyceryl esters thereof, and mixtures thereof.

In a particularly preferred embodiment of the present invention, the fatty acid material is selected from lauric acid, lauroleic acid, myristic acid, myristoleic acid, pentadecanoic acid, palmitic acid, palmitoleic acid, margaric acid, stearic acid, dihydroxystearic acid, oleic acid, ricinoleic acid, elaidic acid, linoleic acid, alpha-linolenic acid, dihomogamma-linolenic acid, eleostearic acid, licanic acid, arachidonic acid, arachidic acid, eicosenoic acid, behenic acid, erucic acid, lignoceric acid, non-glyceryl esters thereof, and mixtures thereof. Additionally, particularly preferred fatty acid materials include oleic acid, linoleic acid, esters thereof, and mixtures thereof. Preferred non-glyceryl esters of this embodiment include ethyl oleate, ethyl linoleate, and mixtures thereof. As an example, ethyl oleate may be obtained from a variety of sources, including Victorian Chemical Co., Richmond, Victoria Australia; Penta Manufacturing Co., Livingston, N.J.; and Croda, Inc., Parsippany, N.J.

The level of LCFA contained within a given composition will typically be dependent upon the particular dosage form selected for use, particularly wherein such level is expressed as a weight percentage relative to all components present within the composition. For example, as further described herein, particularly preferred dose forms will include tablets, capsules, liquids, other concentrated orally deliverable forms, foods, beverages, and the like. Typically, a tablet or capsule may comprise a higher level of LCFA, by weight percent of the composition, relative to (for example) a beverage.

For example, certain dosage forms may optionally comprise from about 0.0001% to about 90% of the LCFA, by weight of the composition. In another embodiment, the compositions may comprise from about 0.001% to about 10%, more preferably from about 0.01% to about 5% of the LCFA, all by weight of the composition. In yet a further embodiment, the compositions may comprise from about 0.01% to about 3% of the LCFA, by weight of the composition.

For example, tablet, capsule, or liquid forms of the present compositions (or other like forms) may optionally comprise from about 1% to about 90% of the LCFA, by weight of the composition. As a further example, emulsion or suspension forms of the present compositions may optionally comprise from about 10% to about 40% of the LCFA, more preferably from about 15% to about 30% of the LCFA, by weight of the composition. Additionally, food or beverage forms may often comprise from about 0.0001% to about 10% of the LCFA, by weight of the composition.

Alternatively or additionally, a single dose of the composition used herein may optionally comprise from about 0.001 grams to about 10 grams of LCFA, more preferably from about 0.004 grams to about 1 grams, all per kilogram body weight of the subject to which the composition is administered. As used herein, the single dose refers to the amount of composition that is typically consumed at a given time.

The Bismuth Component

In an optional embodiment herein, a bismuth component may be included in the compositions containing the LCFA, or otherwise utilized in the kits or methods herein. The bismuth component is a bismuth-containing compound, as will be commonly understood in the art. The bismuth component is preferably a salt such as, for example, those selected from bismuth aluminate, bismuth subcarbonate, bismuth subcitrate, bismuth citrate, tripotassium dicitrato bismuthate, bismuth subgalate, bismuth subnitrate, bismuth tartrate, bismuth subsalicylate, and mixtures thereof. Among these, bismuth citrate, bismuth subsalicylate, and mixtures thereof are even more preferred. Bismuth subsalicylate is most preferred for use herein.

These bismuth components are well known in the art. For example, the formulation and use of various bismuth components are also well known and may be commercially available. For example, bismuth subcitrate may be available as DENOL (Gist-Brocades, N.V.), a combination of bismuth aluminate, alginic acid, and magnesium carbonate may be available as NORALAC (North American Pharmaceuticals), bismuth subnitrate may be available as ROTER bismuth (Roter Laboratories), bismuth subcarbonate (among other materials) may be available as FENSOBAR POLVO (USV Pharmaceutical Corporation), and bismuth subsalicylate (among other materials) may be available as PEPTO BISMOL (The Procter & Gamble Company).

In the kits and methods herein, wherein the bismuth component is utilized, the bismuth component may be present in the same composition as the long chain fatty acid composition or, alternatively, a distinct composition. Certain preferred embodiments employ a composition containing both the bismuth component and the long chain fatty acid component.

The level of the bismuth component contained within a given composition will typically be dependent upon the particular dosage form selected for use, particularly wherein such level is expressed as a weight percentage relative to all components present within the composition. For example, as further described herein, particularly preferred dose forms will include tablets, capsules, liquids, other concentrated orally deliverable forms. Foods, beverages, and the like are also feasible. Typically, a tablet, capsule, or liquid may comprise a higher level of bismuth component.

For example, certain compositions containing a bismuth component will optionally contain from about 0.1% to about 50% of the bismuth component, by weight of the composition. In a preferred embodiment, such compositions contain from about 1% to about 20%, alternatively from about 1% to about 5%, and also alternatively from about 2% to about 4% of the bismuth component, all by weight of the composition.

Alternatively or additionally, a single dose of the composition used herein may optionally comprise from about 0.001 grams to about 10 grams of the bismuth component, more preferably from about 0.004 grams to about 1 grams, all per kilogram body weight of the subject to which the composition is administered. As used herein, the single dose refers to the amount of composition that is typically consumed at a given time.

Other Optional Components

The compositions described herein are useful in a wide variety of finished compositions. The compositions are useful as, for example, pharmaceutical compositions, over-the-counter compositions, dietary supplements, medical foods, foods, and beverages, as well as a variety of other compositions.

The compositions used herein may comprise additional optional components to, for example, enhance their performance or to otherwise render the composition more suitable for use as an industrial or consumer product. Non-limiting examples of optional components are described as follows, which may be more or less relevant depending upon the form of the finished composition.

Water may be included in the compositions of the present invention, for example wherein the compositions are food or beverage compositions, or liquid compositions (such as liquid concentrates). As used herein, the term “water” includes the total amount of water present in the composition including water which is derived from, for example, milk protein, fruit juice, or vegetable juice, as well as any added water. Wherein water is utilized, the water is optionally included at, for example, levels from about 1% to about 99.999%, more preferably from about 5% to about 99%. Ready-to-drink beverage compositions will typically comprise at least about 70% water, preferably from about 75% to about 99% water, all by weight of the composition.

The compositions of the present invention may optionally contain an effective amount of one or more sweeteners, including carbohydrate sweeteners and natural or artificial no/low calorie sweeteners. The amount of the sweetener used in the compositions of the present invention typically depends upon the particular sweetener used and the sweetness intensity desired. For no/low calorie sweeteners, this amount varies depending upon the sweetness intensity of the particular sweetener.

One or more coloring agents may optionally be utilized in the compositions of the present invention. For example, natural or artificial colors may be used.

The compositions herein may optionally comprise one or more flavor agents. Preferably, such flavor agents are included in the beverage compositions and are typically selected from fruit juice, fruit flavors, botanical flavors, and mixtures thereof.

The present compositions may optionally comprise a protein component, for example, animal or plant protein. A non-limiting example of such protein includes milk protein. Milk protein comprises a protein selected from whey, casein, and mixtures thereof. The milk protein may be the protein itself, for example as sodium or calcium caseinate, or may be a component comprising the protein and one or more other materials. For example, various milk proteins are known to one of ordinary skill in the art and may be utilized as the protein component herein.

Milk proteins include mammalian or vegetable milks such as, for example, whole milk, skim milk, condensed milk, dry milk powder, milk protein concentrate, milk protein isolate, milk protein hydrosylate, and mixtures thereof. To illustrate, milk protein concentrate is prepared via milk ultrafiltration or other means such that the lactose or salt content is reduced, thereby enhancing the protein content. In dry and condensed milk, water is removed but all other components of milk are substantially maintained. All forms of milk protein can comprise, for example, intact milk protein, milk protein hydrosylate, or any combination thereof.

The compositions herein may be fortified with one or more nutrients, defined herein as one or more vitamins and/or minerals. Non-limiting examples of vitamins include vitamins A, B₁, B₂, B₃, B₆ and B₁₂, folic acid, pantothenic acid, folic acid, C, D, and E. Non-limiting examples of minerals include calcium, iodine, chromium, magnesium, manganese, molybdenum, selenium, phosphorous, magnesium, zinc, iodine, iron, and copper. As an example, any soluble salt of these minerals suitable for inclusion in edible compositions may optionally be used.

The compositions may comprise one or more fibers. Fibers are well-known in the art and include complex carbohydrates resistant to digestion by mammalian enzymes, such as the carbohydrates found in plant cell walls and seaweed, and those produced by microbial fermentation. Examples of these complex carbohydrates are oat fibers, brans, celluloses, hemicelluloses, pectins, gums and mucilages (e.g., guar gum and gum arabic), seaweed extract, carrageenan, and biosynthetic gums. Sources of the cellulosic fiber include vegetables, fruits, seeds, cereals, and man-made fibers (for example, by bacterial synthesis). Commercial fibers such as purified plant cellulose, or cellulose flour, can also be used. Naturally occurring fibers include fiber from whole citrus peel, citrus albedo, sugar beets, citrus pulp and vesicle solids, apples, apricots, and watermelon rinds.

### Methods of the Present Invention

The methods of the present invention comprise administering a composition as described herein to a mammal, preferably a human or companion animal, for treatment of a gastrointestinal condition. Preferably, the administration is oral administration.

As used herein, the term “gastrointestinal condition” means any infection, disease, or other disorder, whether acute or chronic, of the upper or lower gastrointestinal tract. Non-limiting examples of such conditions include diarrhea, rapid intestinal transit, dumping syndrome, weight loss, distention, steatorrhea, malnutrition, postgastrectomy syndrome, short bowel syndrome, Chron's disease, gastrointestinal peptide tumors, irritable bowel syndrome, irritable bowel disease, abdominal pain, abdominal cramping, and combinations thereof. Diarrhea can include, for example, occasional diarrhea, chronic diarrhea, AIDS-associated diarrhea, diabetes-associated diarrhea, postvagotomy diarrhea, bariatric surgery-associated diarrhea, tube-feeding related diarrhea, secretory diarrhea, carcinoid syndrome-associated diarrhea, thyroid-associated diarrhea, bacterial overgrowth-associated diarrhea, gastrinoma-associated diarrhea, antibiotic-associated diarrhea, irritable bowel syndrome-associated diarrhea, maldigestion or malabsorption-associated diarrhea, idiopathic primary gastrointestinal motility disorder-associated diarrhea, colitis-associated diarrhea, surgery-associated diarrhea, infection-associated diarrhea, and the like.

As used herein, “treatment” of such gastrointestinal condition includes any one or more of curing, alleviating, ameliorating, inhibiting symptoms or the condition or the condition itself, or any like benefit with respect to the gastrointestinal condition.

As used herein, the term “administration,” “administering,” or the like with respect to the mammal means that the mammal is administered is directed to administer or, with reference specifically to “oral administration,” or “orally administering,” ingests or is directed to ingest, one or more compositions of the present invention. Wherein the mammal is directed to ingest one or more of the compositions, such direction may be that which instructs and/or informs the user that use of the composition may and/or will provide one or more general health and/or general physiological benefits including, but not limited to, treatment of a gastrointestinal condition. For example, such direction may be oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” means through words, pictures, symbols, and/or other visible descriptors. Such direction need not utilize the actual words used herein, for example, “treatment”, “gastrointestinal,” “gastrointestinal condition”, or the like, but rather use of words, pictures, symbols, and the like reasonably conveying same or similar meanings are contemplated within the scope of this invention.

The administration may be of a variety of frequencies depending upon the needs or desires of treatment, for example, daily or weekly administration of a composition described herein. Daily administration is particularly preferred, including for example once-daily, twice-daily, and/or three-times-daily administration of a composition. Once daily administration is particularly preferred.

Administration may be on an as-needed basis, for example, in-between meals as symptoms arise. Alternatively or additionally, administration may be concomitant with ingestion of a meal, may be administered between meals, or may serve as a meal replacement. For example, the compositions of this invention may be ingested as a supplement to normal dietetic requirements. As one of ordinary skill in the art will recognize, the composition may be optimized depending upon the time or character of administration.

The composition may be administered as any of a variety of forms, for example, as a food (e.g., a bar), beverage, tablet, capsule, chew, liquid (including emulsions), or another orally administrable form. Wherein the form is a tablet, capsule, liquid or the like, non-limiting techniques for preparing appropriate dosage forms are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

In an optional embodiment of the present invention, the various compositions employed may comprise an enteric delivery system, preferably a small intestinal enteric delivery system. To illustrate, for delivery of the LCFA to the small intestine, the LCFA may be combined with a component having a pH of about 5.5 or greater, such that the LCFA bypasses the stomach unabsorbed, or relatively unabsorbed, and is delivered specifically to desired portions of the small intestine (including proximal or distal). Enteric delivery systems are commonly known.

Dosage will tend to vary, for example, depending upon the mammal which is administered the composition or perhaps the needs of that particular mammal. Typically, a dosage of the composition that is safe and effective will be delivered. Preferred levels of LCFA, and the optional bismuth component, have already been described herein. It is understood that these previously described dosage ranges are by way of example only, and that administration can be adjusted depending on various factors. For example, the specific dosage of the composition which is administered, as well as the duration of treatment, are interdependent. The dosage and treatment regimen will also depend upon such factors as the specific compound used, the treatment indication, the efficacy of the compound, the personal attributes of the mammal (such as, for example, weight, age, gender, and medical condition of the mammal), and compliance with the treatment regimen.

### Kits of the Present Invention

The compositions employed herein may be utilized in kits of the invention. The kits of the present invention comprise one or more compositions described herein together with information which informs a user of the kit, by words, pictures, and/or the like, that use of the kit will provide treatment of a gastrointestinal condition, as has already been described herein. The information may be directed to the benefits of the composition, may include compliance or dosing information, or the like.

In a particularly preferred embodiment, the information is printed on a container holding the composition, as a non-limiting example, a bottle or carton. These preferred kits may be in the form of one container containing the composition or different compositions (wherein distinct compositions are used), or may be obtained as a plurality of containers each containing the composition, or separately containing different compositions (wherein distinct compositions are used).

### EXAMPLES

The following are non-limiting examples of compositions used in accordance with the present methods. The compositions may be prepared using conventional methods. The following examples are provided to illustrate the invention and are not intended to limit the scope thereof in any manner:

Ready-To-Drink Beverage

| | Example I | Example II | Example III |
|---|---|---|---|
| Ingredients | (% w/v) | (% w/v) | (% w/v) |
| Mineral Salts | 0.0062 | 0.0062 | 0.0062 |
| Sodium Hydroxide* | 0.0175 | 0.0175 | 0.0175 |
| Carageenan¹ | 0.0350 | 0.0350 | 0.0350 |
| Cellulose gel² | 0.5200 | 0.5200 | 0.5200 |
| Gum Arabic Low Viscosity | 0.4100 | 0.4100 | 0.4100 |
| Vitamin/Mineral Blend³ | 0.4340 | 0.4340 | 0.4340 |
| Lecithin⁴ | 0.0261 | 0.0261 | 0.0261 |
| Mono & Diglycerides⁵ | 0.0400 | 0.0400 | 0.0400 |
| Ethyl Oleate⁶ | 1.0811 | 2.1505 | 3.2432 |
| Cocoa⁷ | 1.2000 | 1.2000 | 1.200 |
| AF Emulsion Antifoam* | 0.0020 | 0.0020 | 0.0020 |
| Sodium Caseinate⁸ | 0.2990 | 0.2990 | 0.2990 |
| High Heat Nonfat Dry Milk | 6.8000 | 6.8000 | 6.8000 |
| Maltodextrin⁹ | 2.4320 | — | — |
| Granulated Sugar | 3.9500 | 3.9500 | 3.9500 |
| Sucralose Liquid | 0.0539 | 0.0587 | 0.0587 |
| Vanilla Flavor¹⁰ | 0.0015 | 0.0015 | 0.0015 |
| Cream Flavor¹¹ | 0.0015 | 0.0015 | 0.0015 |
| Vanilla Flavor¹² | 0.1000 | 0.1000 | 0.1000 |
| QS Water | | | |
| Total | 100.00 | 100.00 | 100.00 |

*Process aids. Use as needed, up to about level indicated
¹SeaKem CM611 and Viscarin SD389 both from FMC
²Avicel CL-611from FMC
³FT-001106 available from Fortitech
⁴Centrolex-F available from Central Soya Company, Inc.
⁵Myvatex 8-06 available from Quest International USA, Inc.
⁶05-58405 available from Penta Manufacturing Co., or Crodamol EO from Croda Inc.
⁷DeZaan D-11-A available from ADM
⁸Available from DMV International
⁹Maltodextrin 10 DE (M-100) available from Grain Processing Corporation
¹⁰Flavor #14896 available from David Michael and Co., Inc.
¹¹Flavor #1967 available from David Michael and Co., Inc.
¹²Flavor VA03 available from Virginia Dare Extract Co., Inc.

### Example IV

Confectionery Bar

| Material | % w/w |
|---|---|
| High Fructose Corn Syrup | 18.0 |
| Honey-Clover | 18.0 |
| Defatted Peanut Flour | 5.0 |
| Isolated Soy Protein Isolate¹ | 28.2 |
| Nonfat Dry Milk² | 10.0 |
| Brown Sugar | 6.0 |
| Ethyl Oleate³ | 14.0 |
| Natural Orange⁴ | 0.2 |
| Fatty Acid Masker⁵ | 0.1 |
| Natural Peanut Flavor⁶ | 0.1 |
| Natural Honey Flavor⁷ | 0.1 |
| Vanilla Custard Flavor⁸ | 0.1 |
| Soy Lecithin⁹ | 0.2 |
| Total | 100.0 |

¹Supro 661 available from Protein Technologies International
²Low Heat type available from Agrimark
³Crodamol EO available from Croda, Inc., or 05-58405 from Penta Manufacturing Co.
⁴Flavor 57.458/AP SD available from Firmenich
⁵Flavor 348118 available from Firmenich
⁶Flavor 57.304/T available from Firmenich
⁷Flavor 598.513/8 available from Firmenich
⁸Flavor HC600-137 available from P&G
⁹Alcolec S available from American Lecithin Company

### Example V

Molded Tablet

Melt ethyl linoleate and polyoxyl 40 hydrogenated castor oil and combine in a ratio of 4:1 respectively. Pour the combination or melt into appropriately sized tablet molds wherein each tablet contains about 0.5 g palmitic acid. Cool the molds until solidified and remove tablets from the molds.

### Example VI

Soft Gelatin Capsule

Encapsulate ethyl oleate in a #20 oblong soft gelatin capsule using the standard soft-gelatin ribbon/die process such that each capsule contains about 1 gram of the ethyl oleate.

### Example VII

Hard Gelatin Capsule

Combine ethyl oleate and fumed silica in a ratio of 1:1 using a high shear mixer. Encapsulate the resulting mixture into Size 00 hard gelatin capsules using standard high speed capsule filling machine such that each capsule contains about 0.4 grams of the ethyl oleate.

### Example VIII

Pharmaceutical Emulsion

| Component | Wt. Pct. |
|---|---|
| Ethyl Oleate | 20.0 |
| Triglyceryl Monooleate | 3.0 |
| Polysorbate 80 | 2.0 |
| Flavor | 0.5 |
| Sodium Saccharin | 0.2 |
| Purified Water | q.s |
| Total | 100.0 |

1. A method of treating a gastrointestinal condition in a mammal in need of such treatment, wherein the method comprises administering to the mammal a composition comprising a component selected from the group consisting of non-glyceryl esters of a long chain fatty acid, long chain fatty acid salts, and combinations thereof.

2. The method according to claim 1 wherein the composition comprises the non-glyceryl ester of a long chain fatty acid which is selected from the group consisting of methanol esters, ethanol esters, n-propanol esters, iso-propanol esters, n-butanol esters, iso-butanol esters, and mixtures thereof.

3. The method according to claim 2 wherein the non-glyceryl ester of the long chain fatty acids comprises a fatty acid chain having from about 12 to about 24 carbon atoms.

4. The method according to claim 3 wherein composition comprises from about 0.001% to about 10% of the non-glyceryl ester of a long chain fatty acid, by weight of the composition.

5. The method according to claim 4 wherein the non-glyceryl ester of a long chain fatty acid is a non-glyceryl ester of a long chain fatty acid selected from the group consisting of lauric acid, lauroleic acid, myristic acid, myristoleic acid, pentadecanoic acid, palmitic acid, palmitoleic acid, margaric acid, stearic acid, dihydroxystearic acid, oleic acid, ricinoleic acid, elaidic acid, linoleic acid, alpha-linoleic acid, dihomogamma-linoleic acid, eleostearic acid, licanic acid, arachidonic acid, arachidic acid, eicosenoic acid, eicosapentaenoic acid, behenic acid, erucic acid, docosahexaenoic acid, lignoceric acid, and mixtures thereof.

6. The method according to claim 5 wherein the gastrointestinal condition is selected from the group consisting of diarrhea, rapid intestinal transit, dumping syndrome, weight loss, distention, steatorrhea, malnutrition, postgastrectomy syndrome, short bowel syndrome, Chron's disease, gastrointestinal peptide tumors, irritable bowel syndrome, irritable bowel disease, abdominal pain, abdominal cramping, and combinations thereof.

7. The method according to claim 6 wherein the gastrointestinal condition is diarrhea.

8. The method according to claim 7 wherein the non-glyceryl ester of a long chain fatty acid is selected from the group consisting of methyl oleate, ethyl oleate, n-propyl oleate, iso-propyl oleate, n-butyl oleate, iso-butyl oleate, methyl linoleate, ethyl linoleate, n-propyl linoleate, iso-propyl linoleate, n-butyl linoleate, iso-butyl linoleate, and mixtures thereof.

9. The method according to claim 8 wherein the non-glyceryl ester of a long chain fatty acid is ethyl oleate.

10. A method of treating a gastrointestinal condition in a mammal in need of such treatment, wherein the method comprises administering to the mammal a bismuth component and a long chain fatty acid component.

11. A kit comprising:
(a) a composition comprising a component selected from the group consisting of non-glyceryl esters of a long chain fatty acid, long chain fatty acid salts, and combinations thereof; and
(b) information that the composition is useful for the treatment of a gastrointestinal condition in mammals.

12. The kit according to claim 11 wherein the composition comprises the non-glyceryl ester of a long chain fatty acids which is selected from the group consisting of methanol esters, ethanol esters, n-propanol esters, iso-propanol esters, n-butanol esters, iso-butanol esters, and mixtures thereof.

13. The kit according to claim 12 wherein the non-glyceryl esters of the long chain fatty acids comprises a fatty acid chain having from about 12 to about 24 carbon atoms.

14. The kit according to claim 13 wherein composition comprises from about 0.001% to about 10% of the non-glyceryl ester of a long chain fatty acid, by weight of the composition.

15. The kit according to claim 14 wherein the non-glyceryl ester of a long chain fatty acid is a non-glyceryl ester of a long chain fatty acid selected from the group consisting of lauric acid, lauroleic acid, myristic acid, myristoleic acid, pentadecanoic acid, palmitic acid, palmitoleic acid, margaric acid, stearic acid, dihydroxystearic acid, oleic acid, ricinoleic acid, elaidic acid, linoleic acid, alpha-linoleic acid, dihomogamma-linoleic acid, eleostearic acid, licanic acid, arachidonic acid, arachidic acid, eicosenoic acid, eicosapentaenoic acid, behenic acid, erucic acid, docosahexaenoic acid, lignoceric acid, and mixtures thereof.

16. The kit according to claim 15 wherein the gastrointestinal condition is selected from the group consisting of diarrhea, rapid intestinal transit, dumping syndrome, weight loss, distention, steatorrhea, malnutrition, postgastrectomy syndrome, short bowel syndrome, Chron's disease, gastrointestinal peptide tumors, irritable bowel syndrome, irritable bowel disease, abdominal pain, abdominal cramping, and combinations thereof.

17. The kit according to claim 16 wherein the gastrointestinal condition is diarrhea.

18. The kit according to claim 17 wherein the non-glyceryl ester of a long chain fatty acid is selected from the group consisting of methyl oleate, ethyl oleate, n-propyl oleate, iso-propyl oleate, n-butyl oleate, iso-butyl oleate, methyl linoleate, ethyl linoleate, n-propyl linoleate, iso-propyl linoleate, n-butyl linoleate, iso-butyl linoleate, and mixtures thereof.

19. The kit according to claim 18 wherein the non-glyceryl ester of a long chain fatty acid is ethyl oleate.

20. A kit comprising:
(a) a long chain fatty acid component;
(b) a bismuth component; and
(b) information that the composition is useful for the treatment of a gastrointestinal condition in mammals.

21. A composition comprising:
(a) a long chain fatty acid component; and
(b) a bismuth component.

22. The composition according to claim 21 wherein the long chain fatty acid component is selected from the group consisting of lauric acid, lauroleic acid, myristic acid, myristoleic acid, pentadecanoic acid, palmitic acid, palmitoleic acid, margaric acid, stearic acid, dihydroxystearic acid, oleic acid, ricinoleic acid, elaidic acid, linoleic acid, alpha-linoleic acid, dihomogamma-linoleic acid, eleostearic acid, licanic acid, arachidonic acid, arachidic acid, eicosenoic acid, eicosapentaenoic acid, behenic acid, erucic acid, docosahexaenoic acid, lignoceric acid, non-glyceryl esters of any of the foregoing, and mixtures thereof.

23. The composition according to claim 22 wherein the long chain fatty acid component is selected from the group consisting of oleic acid, linoleic acid, methyl oleate, ethyl oleate, n-propyl oleate, iso-propyl oleate, n-butyl oleate, iso-butyl oleate, methyl linoleate, ethyl linoleate, n-propyl linoleate, iso-propyl linoleate, n-butyl linoleate, iso-butyl linoleate, and mixtures thereof.

24. The composition according to claim 23 comprising from about 0.001% to about 10% of the long chain fatty acid component, by weight of the composition.

25. The composition according to claim 24 comprising from about 0.1% to about 50% of the bismuth component, by weight of the composition.

26. The composition according to claim 25 wherein the bismuth component is selected from the group consisting of bismuth aluminate, bismuth subcarbonate, bismuth subcitrate, bismuth citrate, tripotassium dicitrato bismuthate, bismuth subgalate, bismuth subnitrate, bismuth tartrate, bismuth subsalicylate, and mixtures thereof.

27. The composition according to claim 26 wherein the long chain fatty acid component is selected from the group consisting of ethyl oleate, oleic acid, and mixtures thereof.

28. The composition according to claim 27 wherein the bismuth component is bismuth subsalicylate.
